(19) United States
(12) Patent Application Publication
(10) Pub. No.: US 2022/0167903 A1
(43) Pub. Date: Jun. 2, 2022

(54) METHODS AND SYSTEMS TO MANAGE PRESENTATION OF REPRESENTATIVE CARDIAC ACTIVITY SEGMENTS FOR CLUSTERS OF SUCH SEGMENTS

(71) Applicant: Pacesetter, Inc., Sylmar, CA (US)

(72) Inventors: Kevin J. Davis, Thousand Oaks, CA (US); Fujian Qu, San Jose, CA (US); Nima Badie, Oakland, CA (US); Fady Dawoud, Studio City, CA (US); Leyla Sabet, Los Angeles, CA (US)

(21) Appl. No.: 17/399,605

(22) Filed: Aug. 11, 2021

Related U.S. Application Data
(60) Provisional application No. 63/119,099, filed on Nov. 30, 2020.

(51) Int. Cl. A61B 5/35; A61B 5/00

(52) U.S. Cl. CPC A61B 5/35; A61B 5/742; A61B 5/686; A61B 5/7264

(57) ABSTRACT

Methods and systems are provided for managing presentation of cardiac activity signals. The methods and systems obtain device classified (DC) data sets generated by an implantable medical device (IMD), the DC data sets including a corresponding cardiac activity (CA) segment from an episode identified by the IMD; compare the CA segments, associated with different episodes, to one another to identify a level of similarity therebetween; separate the CA segments into at least first and second clusters based on the level of similarity; designate a first representative CA segment from the first cluster to be representative of the CA segments in the first cluster; and designate a second representative CA segment from the second cluster to be representative of the CA segments in the second cluster; and a display to present the first and second representative CA segments as representative of the first and second clusters.

### RELATED APPLICATION

The present application claims priority to U.S. Provisional Application No. 63/119,099, Titled “METHODS AND SYSTEMS TO MANAGE PRESENTATION OF REPRESENTATIVE CARDIAC ACTIVITY SEGMENTS FOR CLUSTERS OF SUCH SEGMENTS” which was filed on 30 Nov. 2020, the complete subject matter of which is expressly incorporated herein by reference in their entirety.

### FIELD OF THE INVENTION

Embodiments herein relate generally to managing presentation of cardiac activity (CA) segments by limiting presentation to representative CA segments for clusters.

### BACKGROUND OF THE INVENTION

Today, numerous arrhythmia detection processes are implemented within various types of implantable medical devices (IMDs). One type of IMD is an implantable cardiac monitor (ICM) that detects arrhythmias based on various criteria, such as irregularities and variation patterns in R-wave to R-wave (RR) intervals. In some embodiments, the arrhythmia detection process steps beat by beat through cardiac activity (CA) signals and analyzes the characteristics of interest, such as RR intervals over a period of time. An arrhythmia episode is declared based on the characteristics of interest, such as when the RR interval pattern for the suspect beats is sufficiently irregular and dissimilar from RR interval patterns for sinus beats. In some instances, an arrhythmia episode may continue over a relatively long period of time (e.g., 30 minutes, 1 hour), and a patient may experience numerous arrhythmia episodes between points in time in which the IMD is able to transmit stored EGM signals to an external device or data server. For example, over the course of a day, week, month, a patient may experience multiple atrial fibrillation (AF) episodes, with each AF episode lasting 30 minutes or more. It is desirable for the IMD to record EGM signals in connection with each AF episode. However, due to memory constraints, it is not practical for the IMD to store the entire EGM signals for numerous 30-minute AF episodes. Accordingly, the IMD typically stores a short segment of EGM signals, such as 30 seconds at the beginning of each AF episode, even though the AF episode may last longer. The IMD may also store a short segment of EGM signal at the end of each AF episode. The IMD is then able to link these short segments of EGM signals with all or most of the AF episodes experienced by the patient between time periods in which the IMD is able to transmit the EGM signals to an external device or data server.

Some implantable medical devices, particularly implantable cardiac monitors (ICMs), may store and subsequently transmit a large number of stored EGMs (SEGMs) to a remote monitoring server. Each SEGM corresponds to a different arrhythmia episode detected by the device. Accordingly, the clinician will review the SEGMs associated with the arrhythmia episodes, in order to confirm the device detected episodes are true arrhythmic events and to determine treatment options for optimal patient management. For patients with frequently detected arrhythmia episodes, the IMD will generate a correspondingly large number of SEGMs which places a large burden on the clinician to review. To reduce the SEGM data review burden, some systems employ a “key episode” approach which presents to the clinician only limited number SEGMs that meet certain criteria. For example, common criteria include identifying SEGMs associated with the arrhythmia episode having the “fastest rate”, “longest duration”, or “earliest onset”, or a combination thereof. For example, when and IMD downloads a collection of 10-20 SEGMs for a corresponding number of 10-20 arrhythmia episodes, the clinician may be presented with only the one or two SEGMs associated with the arrhythmia having the fastest heart rate, or the arrhythmia that was sustained for the longest duration, or otherwise.

The foregoing example has two limitations. First, the selection criteria do not guarantee that the “key episodes” are true arrhythmic events. It may miss or delay arrhythmia diagnosis. Second, the selection criteria are applied on episodes between data upload operations (e.g., to external device and data server). For example, the device may detected two episodes on day 1 and another two episodes on day 2, 3, etc. Even if the episode on day 2, 3, etc. are identical to the episodes on day 1, they are still uploaded. Therefore, the ability to reduce EGM volume by this approach is limited.

Also, the clinician is not presented with the SEGMs for arrhythmia episodes that have a relatively slower heart rate or shorter duration, even though such arrhythmia episodes may warrant clinical review. Instead, in order for the clinician to be confident that the clinician has reviewed SEGMs in connection with every different type of arrhythmia episode experienced by the patient, the clinician is required to step through every SEGM that was downloaded, again placing an undue burden on the clinician.

A need remains to reduce the burden placed on clinicians for reviewing SEGM signals, and in particular in connection with large volumes of similar SEGM signals.

### SUMMARY

In accordance with embodiments herein, methods and systems are described that utilize a “similarity” measure as a filter for CA signals that exhibit the same underlying physiological or environmental mechanism.

In accordance with embodiments herein, a system is provided for managing presentation of cardiac activity signals, comprising: memory to store specific executable instructions; one or more processors configured to execute the specific executable instructions to: obtain device classified (DC) data sets generated by an implantable medical device (IMD), the DC data sets including a corresponding cardiac activity (CA) segment from an episode identified by the IMD; compare the CA segments, associated with different episodes, to one another to identify a level of similarity therebetween; separate the CA segments into at least first and second clusters based on the level of similarity; designate a first representative CA segment from the first cluster to be representative of the CA segments in the first cluster; and designate a second representative CA segment from the second cluster to be representative of the CA segments in the second cluster; and a display to present the first and second representative CA segments as representative of the first and second clusters.

Additionally or alternatively, the first representative CA segment is associated with a first episode, the first cluster includes additional CA segments, associated with additional episodes, the additional CA segments falling within the level of similarity to the first representative CA segment. Additionally or alternatively, the first cluster includes additional CA segments are redundant as to shape, morphology and/or other characteristic of interest of the first representative CA segment, the one or more processors further configured to not display the additional CA segments. Additionally or alternatively, the one or more processors are configured to calculate a power spectral density (PSD) for each of the CA segments and to compare the PSDs for respective ones of the CA segments to identify the level of similarity. Additionally or alternatively, the one or more processors are configured to compare the CA signals by utilizing at least one of a cross correlation technique or a power spectral estimate. Additionally or alternatively, the first and second clusters include prior first and second sets of CA segments, the one or more processors further configured to compare a current CA segment to the prior first set of CA segments, and if the level of similarity does not satisfy the threshold, to then compare the current CA segments to the prior second set of CA segments. Additionally or alternatively, the one or more processors are further configured to select, as the first representative CA segment, a one of the CA segments in the first cluster that at least one of: i) was first assigned to the first cluster, ii) associated with the longest episode duration, iii) was the most recently assigned to the cluster, or iv) exhibits a select level of similarity to a remainder of the CA segments in the first cluster.

Additionally or alternatively, the system includes a sensor to collect CA signals, the DC data set based on the CA signals, the CA signals indicative of at least one of impedance, electrical or mechanical activity by one or more heart chambers or by a local region within the heart. Additionally or alternatively, the CA signals include at least one of EGM signals or heart sound (HS) based CA signals, the HS based CA signals indicative of one or more of the S1, S2, S3 or S4 heart sounds.

In accordance with embodiments herein, a computer implemented method is provided, comprising: under control of one or more processors configured with specific executable instructions, obtaining device classified (DC) data sets generated by an implantable medical device (IMD), each of the DC data sets including a cardiac activity (CA) segment from an episode identified by the IMD; comparing the CA segments, associated with different episodes, to one another to identify a level of similarity therebetween; separating the CA segments into at least first and second clusters based on the level of similarity; designating a first representative CA segment from the first cluster to be representative of the CA segments in the first cluster; and designating a second representative CA segment from the second cluster to be representative of the CA segments in the second cluster; and presenting the first and second representative CA segments as representative of the first and second clusters.

Additionally or alternatively, the first representative CA segment is associated with a first episode, the first cluster includes additional CA segments, associated with additional episodes, the additional CA segments falling within the level of similarity to the first representative CA segment. Additionally or alternatively, the first cluster includes additional CA segments are redundant as to shape, morphology and/or other characteristic of interest of the first representative CA segment, the method further comprising not displaying the additional CA signals. Additionally or alternatively, the method further comprises calculating a power spectral density (PSD) for each of the CA segments and comparing the PSDs for respective ones of the CA segments to identify the level of similarity. Additionally or alternatively, the comparing the CA signals utilizes at least one of a cross correlation technique or a power spectral estimate. Additionally or alternatively, the first and second clusters include prior first and second sets of CA segments, the method further comprising comparing a current CA segment to the prior first set of CA segments, and if the level of similarity does not satisfy the threshold, then comparing the current CA segments to the prior second set of CA segments. Additionally or alternatively, the method further comprises selecting, as the first representative CA segment, a one of the CA segments in the first cluster that at least one of: i) was first assigned to the first cluster, ii) associated with the longest episode duration, iii) was the most recently assigned to the cluster, or iv) exhibits a select level of similarity to a remainder of the CA segments in the first cluster.

Additionally or alternatively, the method utilizes a sensor to collect CA signals, the DC data set based on the CA signals, the CA signals indicative of at least one of impedance, electrical or mechanical activity by one or more heart chambers or by a local region within the heart. Additionally or alternatively, the CA signals include at least one of EGM signals or heart sound (HS) based CA signals, the HS based CA signals indicative of one or more of the S1, S2, S3 or S4 heart sounds.

### BRIEF DESCRIPTION OF THE DRAWINGS

FIG. 1 illustrates an ICM intended for subcutaneous implantation at a site near the heart in accordance with embodiments herein.

FIG. 2 shows a block diagram of the ICM formed in accordance with embodiments herein.

FIG. 3 shows a high-level overview of a system formed in accordance with embodiments herein.

FIG. 4 illustrates a process for clustering similar device classified data sets (DCA data sets and/or DCNS data sets) in accordance with embodiments herein.

FIG. 5 illustrates a process for calculating power spectral density estimates for a CA segment from a single DC data set in accordance with embodiments herein.

FIG. 6 illustrates a graphical example of a manner in which CA signals may be converted to the frequency domain.

FIG. 7A illustrates first and second CA segments representing CA signals in the time domain.

FIG. 7B illustrates first and second clusters, into which prior CA segments have been assigned.

FIG. 7C illustrates a circumstance in which the current CA segment is identified to be similar to at least one of the prior CA segments the current CA segment is assigned to the second cluster.

FIG. 7D illustrates a circumstance in which the current CA segment is not similar to CA segments in any prior cluster and thus a new clusters generated.

FIG. 8 illustrates a distributed processing system in accordance with embodiments herein.

FIG. 9 illustrates a system level diagram indicating potential devices and networks that utilize the methods and systems herein.

### DETAILED DESCRIPTION

The terms “abnormal,” or “arrhythmic” are used to refer to events, features, and characteristics of, or appropriate to, a un-healthy or abnormal functioning of the heart.

The terms “beat” and “cardiac event” are used interchangeably and shall include both normal or abnormal events.

The terms “cardiac activity signal”, “cardiac activity signals”, “CA signal” and “CA signals” (collectively “CA signals”) are used interchangeably throughout to refer to measured signals indicative of cardiac activity by a region or chamber of interest. For example, the CA signals may be indicative of impedance, electrical or mechanical activity by one or more chambers (e.g., left or right ventricle, left or right atrium) of the heart and/or by a local region within the heart (e.g., impedance, electrical or mechanical activity at the AV node, along the septal wall, within the left or right bundle branch, within the purkinje fibers). The cardiac activity may be normal/healthy or abnormal/arrhythmic. An example of CA signals includes EGM signals. Electrical based CA signals refer to an analog or digital electrical signal recorded by two or more electrodes, where the electrical signals are indicative of cardiac activity. Heart sound (HS) based CA signals refer to signals output by a heart sound sensor such as an accelerometer, where the HS based CA signals are indicative of one or more of the S1, S2, S3 and/or S4 heart sounds. Impedance based CA signals refer to impedance measurements recorded along an impedance vector between two or more electrodes, where the impedance measurements are indicative of cardiac activity.

The term “CA segment” refers to the CA signals collected for a predetermined interval, such as a period of time, a number of beats and the like. By way of example, a CA segment may represent a 30 second strip of EGM signals.

The term “COI” refers to a character of interest. Nonlimiting examples of characters of interest within CA signals include an R-wave, P-wave, T-wave, S1 heart sound, S2 heart sound, S3 heart sound or S4 heart sound. A character of interest may correspond to a peak, average, mean or other statistical parameter of an individual R, P, R or T-wave peak, S1 heart sound, S2 heart sound, S3 heart sound or S4 heart sound and the like. Non-limiting examples of COI from CA signals collected at an individual electrode(s) include a sensed event (e.g., an intrinsic event or evoked response).

The terms “device classified arrhythmia data set” and “DCA data set” are used interchangeably and shall mean a data set that includes i) CA signals collected in response to a determination by an IMD that the CA signals are indicative of an arrhythmia of interest and ii) one or more device documented markers related to one or more features of interest in the CA signals that in whole or in part were utilized by the IMD in connection with the determination of the arrhythmia of interest.

The terms “device classified normal sinus data set” and “DCNS data set” are used interchangeably and shall mean a data set that includes i) CA signals collected in response to a determination by an IMD that the CA signals are indicative of a normal sinus rhythm or an event of interest within a chamber and ii) one or more device documented markers related to one or more features of interest in the CA signals that in whole or in part were utilized by the IMD in connection with the determination of the normal sinus rhythm. For example, a DCNS data set may be generated by a leadless device in a chamber of the heart where the DCNS data set includes CA signals corresponding to heart sounds detected in the same chamber for a different chamber. As a further example, a leadless device in the RV may “listen” for heart sounds indicative of contraction of the RA. The CA signal may correspond to the heart sounds for RA contraction.

The term “DC data set” shall refer generally to both DCA data sets and/or DCNS data sets.

The term “device documented marker” refers to markers that are generated by an IMD to characterize one or more features of interest within respective CA signals. Markers may be declared based on numerous criteria, such as signal processing, feature detection and arrhythmia detection software and hardware within and/or operating on the implantable cardiac monitor and/or implantable medical device.

The terms “similar”, “similarity” and “level of similarity”, as used in connection with describing CA segments, CA signals and DC data sets, shall mean having a same or substantially same shape, morphology and/or other characteristics of interest, and to the extent differences exist between the shape, morphology and/or other characteristic of interest, such differences are minor, not physiologically significant and/or would not change a clinicians diagnosis and/or decision regarding what action to take.

The term “marker” shall mean data and/or information identified from CA signals that may be presented as graphical and/or numeric indicia indicative of one or more features within the CA signals and/or indicative of one or more episodes exhibited by the cardiac events. Markers may be superimposed upon CA signals or presented proximate to, and temporally aligned with, CA signals. Non-limiting examples of markers may include R-wave markers, noise markers, activity markers, interval markers, refractory markers, P-wave markers, T-wave markers, PVC markers, sinus rhythm markers, AF markers and other arrhythmia markers. As a further nonlimiting example, basic event markers may include “AF entry” to indicate a beginning of an AF event, “in AF” to indicate that AF is ongoing, “AF exit” to indicate that AF has terminated, “T” to indicate a tachycardia beat, “B” to indicate a bradycardia beat, “A” to indicate an asystole beat, “VS” to indicate a regular sinus beat, “Tachy” to indicate a tachycardia episode, “Brady” to indicate a Bradycardia episode, “Asystole” to indicate an asystole episode, “Patient activated” to indicate a patient activated episode. An activity marker may indicate activity detected by activity sensor during the CA signal. Noise markers may indicate entry/start, ongoing, recovery and exit/stop of noise. Markers may be presented as symbols, dashed lines, numeric values, thickened portions of a waveform, and the like. Markers may represent events, intervals, refractory periods, ICM activity, and other algorithm related activity. For example, interval markers, such as the R-R interval, may include a numeric value indicating the duration of the interval. The AF markers indicate atrial fibrillation rhythmic.

The term “machine learning” shall mean an artificial intelligence algorithm that learns from various automatic or manual inputs, such as features of interest, prior device classified arrhythmias, observations and/or data. The machine learning algorithm is adjusted over multiple iterations based on the features of interest, prior device classified arrhythmias, observations and/or data. For example, the machine learning algorithm is adjusted by supervised learning, unsupervised learning, and/or reinforcement learning. Non-limiting examples of machine learning algorithms are a convolutional neural network, gradient boosting random forest, decision tree, K-means, deep learning, artificial neural network, and/or the like.

The terms “normal” and “sinus” are used to refer to events, features, and characteristics of, or appropriate to, a heart's healthy or normal functioning.

The term “obtain”, as used in connection with data, signals, information and the like, includes at least one of i) accessing memory of an IMD, ICM, external device or remote server where the data, signals, information, etc. are stored, ii) receiving the data, signals, information, etc. over a wireless communications link between the ICM or IMD and a local external device, iii) receiving the data, signals, information, etc. at a remote server over a network connection and/or iv) sensing signals (e.g., CA signals, impedance signals, etc.) between a combination of electrodes provide on or coupled to the ICM or IMD. An obtaining operation, when from the perspective of an ICM or IMD, may include sensing new signals in real time, and/or accessing memory to read stored data, signals, information, etc. from memory within the ICM or IMD. The obtaining operation, when from the perspective of a local external device, includes receiving the data, signals, information, etc. at a transceiver of the local external device where the data, signals, information, etc. are transmitted from an ICM and/or a remote server. The obtaining operation may be from the perspective of a remote server, such as when receiving the data, signals, information, etc. at a network interface from a local external device and/or directly from an ICM. The remote server may also obtain the data, signals, information, etc. from local memory and/or from other memory, such as within a cloud storage environment and/or from the memory of a workstation or clinician external programmer.

The term “real-time” refers to a time frame contemporaneous with occurrence of a normal or abnormal episode. For example, a real-time process or operation would occur during or immediately after (e.g., within minutes or seconds after) a cardiac event, a series of cardiac events, an arrhythmia episode, and the like.

The term “subcutaneous” shall mean below the skin surface but not within the heart and not transvenous.

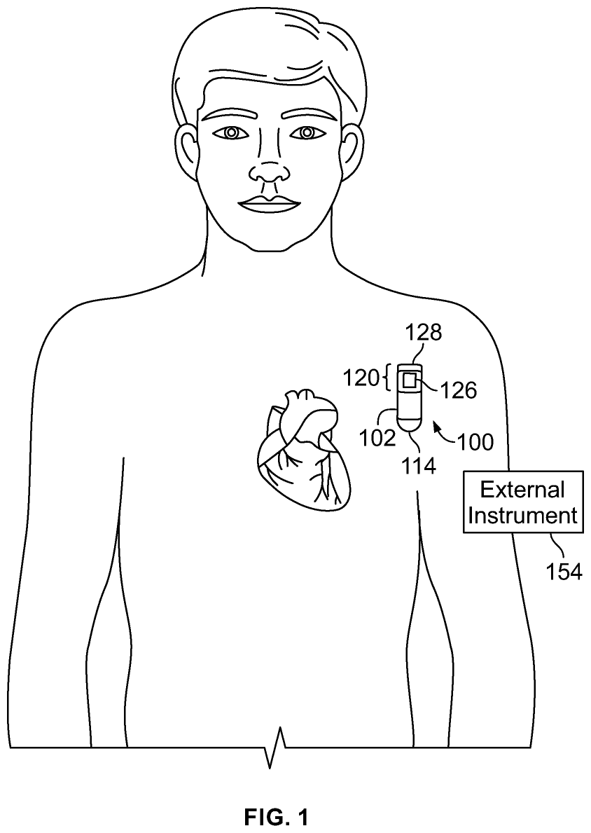

FIG. 1 illustrates an ICM 100 intended for subcutaneous implantation at a site near the heart. The ICM 100 includes a pair of spaced-apart sense electrodes 114, 126 positioned with respect to a housing 102. The sense electrodes 114, 126 provide for detection of far field electrogram signals. Numerous configurations of electrode arrangements are possible. For example, the electrode 114 may be located on a distal end of the ICM 100, while the electrode 126 is located on a proximal side of the ICM 100. Additionally or alternatively, electrodes 126 may be located on opposite sides of the ICM 100, opposite ends or elsewhere. The distal electrode 114 may be formed as part of the housing 102, for example, by coating all but a portion of the housing with a nonconductive material such that the uncoated portion forms the electrode 114. In this case, the electrode 126 may be electrically isolated from the housing 102 electrode by placing it on a component separate from the housing 102, such as the header 120. Optionally, the header 120 may be formed as an integral portion of the housing 102. The header 120 includes an antenna 128 and the electrode 126. The antenna 128 is configured to wirelessly communicate with an external device 154 in accordance with one or more predetermined wireless protocols (e.g., Bluetooth, Bluetooth low energy, Wi-Fi, etc.). The housing 102 includes various other components such as: sense electronics for receiving signals from the electrodes, a microprocessor for processing the signals in accordance with algorithms, such as the AF detection algorithm described herein, a loop memory for temporary storage of CA data, a device memory for long-term storage of CA data upon certain triggering events, such as AF detection, sensors for detecting patient activity and a battery for powering components.

In at least some embodiments, the ICM 100 is configured to be placed subcutaneously utilizing a minimally invasive approach. Subcutaneous electrodes are provided on the housing 102 to simplify the implant procedure and eliminate a need for a transvenous lead system. The sensing electrodes may be located on opposite sides of the device and designed to provide robust episode detection through consistent contact at a sensor-tissue interface. The ICM 100 may be configured to be activated by the patient or automatically activated, in connection with recording subcutaneous ECG signals.

The ICM 100 senses far field, subcutaneous CA signals, processes the CA signals to detect arrhythmias and if an arrhythmia is detected, automatically records the CA signals in memory for subsequent transmission to an external device. The CA signal processing and AF detection is provided for, at least in part, by algorithms embodied in or implemented by the microprocessor. The ICM 100 includes one or more processors and memory that stores program instructions directing the processors to implement AF detection utilizing an on-board R-R interval irregularity (ORI) process that analyzes cardiac activity signals collected over one or more sensing channels.

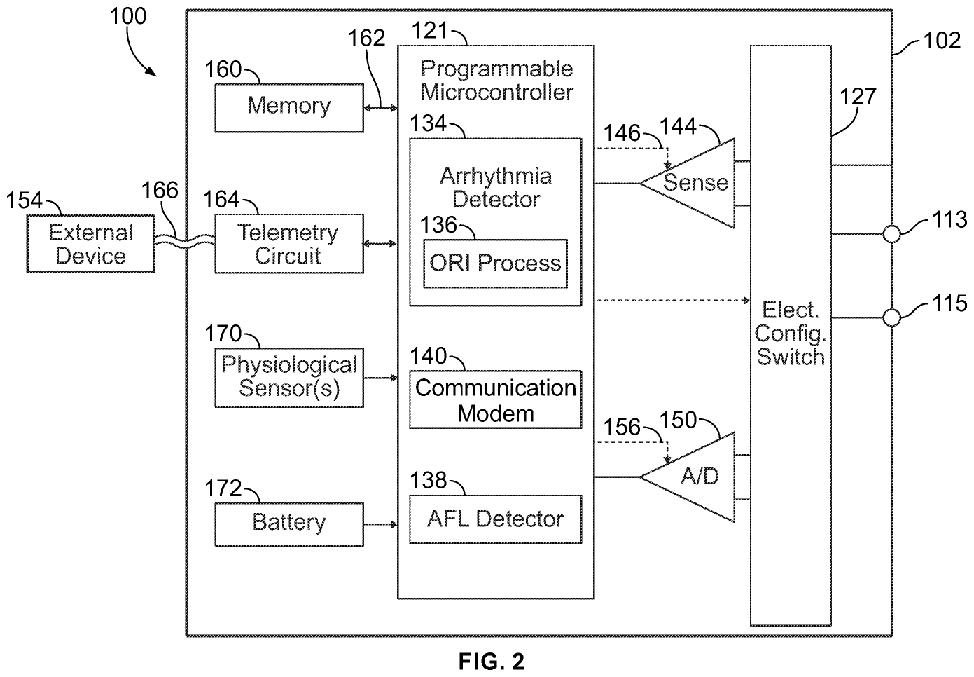

FIG. 2 shows a block diagram of the ICM 100 formed in accordance with embodiments herein. The ICM 100 may be implemented to monitor ventricular activity alone, or both ventricular and atrial activity through sensing circuit. The ICM 100 has a housing 102 to hold the electronic/computing components. The housing 102 (which is often referred to as the “can”, “case”, “encasing”, or “case electrode”) may be programmably selected to act as an electrode for certain sensing modes. Housing 102 further includes a connector (not shown) with at least one terminal 113 and optionally additional terminals 115. The terminals 113, 115 may be coupled to sensing electrodes that are provided upon or immediately adjacent the housing 102. Optionally, more than two terminals 113, 115 may be provided in order to support more than two sensing electrodes, such as for a bipolar sensing scheme that uses the housing 102 as a reference electrode. Additionally or alternatively, the terminals 113, 115 may be connected to one or more leads having one or more electrodes provided thereon, where the electrodes are located in various locations about the heart. The type and location of each electrode may vary.

The ICM 100 includes a programmable microcontroller 121 that controls various operations of the ICM 100, including cardiac monitoring. Microcontroller 121 includes a microprocessor (or equivalent control circuitry), RAM and/or ROM memory, logic and timing circuitry, state machine circuitry, and I/O circuitry. The microcontroller 121 also performs the operations described herein in connection with collecting cardiac activity data and analyzing the cardiac activity data.

A switch 127 is optionally provided to allow selection of different electrode configurations under the control of the microcontroller 121. The electrode configuration switch 127 may include multiple switches for connecting the desired electrodes to the appropriate I/O circuits, thereby facilitating electrode programmability. The switch 127 is controlled by a control signal from the microcontroller 121. Optionally, the switch 127 may be omitted and the I/O circuits directly connected to the housing electrode 114 and a second electrode 126.

Microcontroller 121 includes an arrhythmia detector 134 that is configured to analyze cardiac activity signals to identify potential arrhythmia episodes (e.g., Tachycardias, Bradycardias, Asystole, Brady pause, atrial fibrillation, etc.). By way of example, the arrhythmia detector 134 may implement an arrhythmia detection algorithm as described in U.S. Pat. No. 8,135,456, the complete subject matter of which is incorporated herein by reference. Although not shown, the microcontroller 121 may further include other dedicated circuitry and/or firmware/software components that assist in monitoring various conditions of the patient's heart and managing pacing therapies. The arrhythmia detector 134 of the microcontroller 121 includes an on-board R-R interval irregularity (ORI) process 136 that detects arrhythmia episodes, such as AF episodes using R-R interval irregularities. The ORI process 136 may be implemented as firmware, software and/or circuits. The ORI process 136 uses a hidden Markov Chains and Euclidian distance calculations of similarity to assess the transitionary behavior of one R-wave (RR) interval to another and compare the patient's RR interval transitions to the known RR interval transitions during atrial fibrillation (AF) and non-AF episodes obtained from the same patient and/or many patients.

The arrhythmia detector 134 analyzes sensed far field CA signals sensed along a sensing vector between a combination of subcutaneous electrodes for one or more beats. The arrhythmia detector 134 identifies one or more features of interest from the CA signals, and based on further analysis of the features of interest determines whether the CA signals are indicative of a normal sinus rhythm or an arrhythmia episode. When an arrhythmia episode is identified, the arrhythmia detector 134 generates one or more DD markers that are temporally aligned with corresponding features of interest in the CA signals. The arrhythmia detector 134 forms a DCA data set associated with the classified arrhythmia episode and stores the DCA data set in the memory of the IMD. The arrhythmia detector 134 iteratively or periodically repeats the analysis of incoming far field CA signals to continuously add DCA data sets for respective arrhythmia episodes, thereby forming a collection of DCA data sets.

The ICM 100 is further equipped with a communication modem (modulator/demodulator) 140 to enable wireless communication. In one implementation, the communication modem 140 uses high frequency modulation, for example using RF, Bluetooth or Bluetooth Low Energy telemetry protocols. The signals are transmitted in a high frequency range and will travel through the body tissue in fluids without stimulating the heart or being felt by the patient. The communication modem 140 may be implemented in hardware as part of the microcontroller 121, or as software/firmware instructions programmed into and executed by the microcontroller 121. Alternatively, the modem 140 may reside separately from the microcontroller as a standalone component. The modem 140 facilitates data retrieval from a remote monitoring network. The modem 140 enables timely and accurate data transfer directly from the patient to an electronic device utilized by a physician.

The ICM 100 includes sensing circuit 144 selectively coupled to one or more electrodes that perform sensing operations, through the switch 127 to detect cardiac activity data indicative of cardiac activity. The sensing circuit 144 may include dedicated sense amplifiers, multiplexed amplifiers, or shared amplifiers. It may further employ one or more low power, precision amplifiers with programmable gain and/or automatic gain control, bandpass filtering, and threshold detection circuit to selectively sense the features of interest. In one embodiment, switch 127 may be used to determine the sensing polarity of the cardiac signal by selectively closing the appropriate switches.

The output of the sensing circuit 144 is connected to the microcontroller 121 which, in turn, determines when to store the cardiac activity data of CA signals (digitized by the A/D data acquisition system 150) in the memory 160. For example, the microcontroller 121 may only store the cardiac activity data (from the ND data acquisition system 150) in the memory 160 when a potential arrhythmia episode is detected. The sensing circuit 144 receives a control signal 146 from the microcontroller 121 for purposes of controlling the gain, threshold, polarization charge removal circuitry (not shown), and the timing of any blocking circuitry (not shown) coupled to the inputs of the sensing circuit.

Optionally, the ICM 100 may include multiple sensing circuits, similar to sensing circuit 144, where each sensing circuit is coupled to two or more electrodes and controlled by the microcontroller 121 to sense electrical activity detected at the corresponding two or more electrodes. The sensing circuit 144 may operate in a unipolar sensing configuration or in a bipolar sensing configuration. Optionally, the sensing circuit 144 may be removed entirely and the microcontroller 121 perform the operations described herein based upon the CA signals from the ND data acquisition system 150 directly coupled to the electrodes.

The ICM 100 further includes an analog-to-digital ND data acquisition system (DAS) 150 coupled to one or more electrodes via the switch 127 to sample cardiac activity signals across any pair of desired electrodes. The data acquisition system 150 is configured to acquire cardiac electrogram (EGM) signals as CA signals, convert the raw analog data into digital data, and store the digital data as CA data for later processing and/or telemetric transmission to an external device 154 (e.g., a programmer, local transceiver, or a diagnostic system analyzer). The data acquisition system 150 is controlled by a control signal 156 from the microcontroller 121. The EGM signals may be utilized as the cardiac activity data that is analyzed for potential arrhythmia episodes. The ACS adjustment and ORI process 136 may be applied to signals from the sensing circuit 144 and/or the DAS 150.

By way of example, the external device 154 may represent a bedside monitor installed in a patient's home and utilized to communicate with the ICM 100 while the patient is at home, in bed or asleep. The external device 154 may be a programmer used in the clinic to interrogate the ICM 100, retrieve data and program detection criteria and other features. The external device 154 may be a handheld device (e.g., smartphone, tablet device, laptop computer, smartwatch and the like) that can be coupled over a network (e.g., the Internet) to a remote monitoring service, medical network and the like. The external device 154 facilitates access by physicians to patient data as well as permitting the physician to review real-time CA signals while collected by the ICM 100.

The microcontroller 121 is coupled to a memory 160 by a suitable data/address bus 162. The programmable operating parameters used by the microcontroller 121 are stored in memory 160 and used to customize the operation of the ICM 100 to suit the needs of a particular patient. Such operating parameters define, for example, detection rate thresholds, sensitivity, automatic features, AF detection criteria, activity sensing or other physiological sensors, and electrode polarity, etc.

In addition, the memory 160 stores the cardiac activity data, as well as the markers and other data content associated with detection of arrhythmia episodes. The operating parameters of the ICM 100 may be non-invasively programmed into the memory 160 through a telemetry circuit 164 in telemetric communication via communication link 166 with the external device 154. The telemetry circuit 164 allows intracardiac electrograms and status information relating to the operation of the ICM 100 (as contained in the microcontroller 121 or memory 160) to be sent to the external device 154 through the established communication link 166. In accordance with embodiments herein, the telemetry circuit 164 conveys the DCA data sets and other information related to arrhythmia episodes to an external device.

The ICM 100 may further include magnet detection circuitry (not shown), coupled to the microcontroller 121, to detect when a magnet is placed over the unit. A magnet may be used by a clinician to perform various test functions of the housing 102 and/or to signal the microcontroller 121 that the external device 154 is in place to receive or transmit data to the microcontroller 121 through the telemetry circuits 164.

The ICM 100 can further include one or more physiologic sensors 170. Such sensors are commonly referred to (in the pacemaker arts) as “rate-responsive” or “exercise” sensors. The physiological sensor 170 may further be used to detect changes in the physiological condition of the heart, or diurnal changes in activity (e.g., detecting sleep and wake states). Signals generated by the physiological sensors 170 are passed to the microcontroller 121 for analysis and optional storage in the memory 160 in connection with the cardiac activity data, markers, episode information and the like. While shown as being included within the housing 102, the physiologic sensor(s) 170 may be external to the housing 102, yet still be implanted within or carried by the patient. Examples of physiologic sensors might include sensors that, for example, activity, temperature, sense respiration rate, pH of blood, ventricular gradient, activity, position/posture, minute ventilation (MV), and so forth. Additionally or alternatively, the physiologic sensor may be implemented as an accelerometer and may be implemented utilizing all or portions of the structural and/or functional aspects of the methods and systems described in U.S. Pat. No. 6,937,900, titled “AC/DC Multi-Axis Accelerometer for Determining A Patient Activity and Body Position;” U.S. application Ser. No. 17/192,961, filed Mar. 5, 2021, (attorney docket 13-0397US1) (client docket 13967US01), titled “SYSTEM FOR VERIFYING A PATHOLOGIC EPISODE USING AN ACCELEROMETER”; U.S. application Ser. No. 16/869,733, filed May 8, 2020, (attorney docket 13-0396US1) (client docket 13964USO1), titled “METHOD AND DEVICE FOR DETECTING RESPIRATION ANOMALY FROM LOW FREQUENCY COMPONENT OF ELECTRICAL CARDIAC ACTIVITY SIGNALS;” U.S. application Ser. No. 17/194,354, filed Mar. 8, 2021, (Attorney docket 13-0395US1) (client docket 13949USO1), titled “METHOD AND SYSTEMS FOR HEART CONDITION DETECTION USING AN ACCELEROMETER;” U.S. application Ser. No. 17/353,172, filed Jun. 21, 2021, (Attorney docket 13-0410US1) (client docket 14039US01), titled “METHOD AND DEVICE FOR CONTROLLING CARDIAC RESYNCHRONIZATION THERAPY BASED ON HEART SOUNDS”, the complete subject matter which is expressly incorporated herein by reference.

A battery 172 provides operating power to all of the components in the ICM 100. The battery 172 is capable of operating at low current drains for long periods of time. The battery 172 also desirably has a predictable discharge characteristic so that elective replacement time can be detected. As one example, the housing 102 employs lithium/silver vanadium oxide batteries. The battery 172 may afford various periods of longevity (e.g., three years or more of device monitoring). In alternate embodiments, the battery 172 could be rechargeable. See for example, U.S. Pat. No. 7,294,108, Cardiac event micro-recorder and method for implanting same, which is hereby incorporated by reference.

The ICM 100 provides a simple to configure data storage option to enable physicians to prioritize data based on individual patient conditions, to capture significant events and reduce risk that unexpected events are missed. The ICM 100 may be programmable for pre- and post-trigger event storage. For example, the ICM 100 may be automatically activated to store 10-120 seconds of CA data prior to an event of interest and/or to store 10-120 seconds of post CA data. Optionally, the ICM 100 may afford patient triggered activation in which pre-event CA data is stored, as well as post event CA data (e.g., pre-event storage of 1-15 minutes and post-event storage of 1-15 minutes). Optionally, the ICM 100 may afford manual (patient triggered) or automatic activation for CA data. Optionally, the ICM 100 may afford additional programming options (e.g., asystole duration, bradycardia rate, tachycardia rate, tachycardia cycle count). The amount of CA data storage may vary based upon the size of the memory 160.

The ICM 100 may provide comprehensive safe diagnostic data reports including a summary of heart rate, in order to assist physicians in diagnosis and treatment of patient conditions. By way of example, reports may include episode diagnostics for auto trigger events, episode duration, episode count, episode date/time stamp and heart rate histograms. The ICM 100 may be configured to be relatively small (e.g., between 2-10 cc in volume) which may, among other things, reduce risk of infection during implant procedure, afford the use of a small incision, afford the use of a smaller subcutaneous pocket and the like. The small footprint may also reduce implant time and introduce less change in body image for patients.

While illustrated as an implantable cardiac monitor (ICM), in other example embodiments the IMD can be a leadless device. Optionally, the leadless device can include a housing, multiple electrodes coupled to the housing, and a pulse generator hermetically contained within the housing and electrically coupled to the electrodes. A pulse generator may be provided and configured for sourcing energy internal to the housing, generating, and delivering electrical pulses to the electrodes. A controller can also be hermetically contained within the housing as part of the pulse generator and communicatively coupled to the electrodes. The controller can control, among other things, recording of physiologic characteristics of interest and/or electrical pulse delivery based on the sensed activity.

Optionally, a first leadless device can be located in the right atrium (RA), while a second leadless device is located in the right ventricle (RV). The leadless devices coordinate the operation therebetween based in part on information conveyed between the leadless devices during operation. The information conveyed between the leadless devices may include, among other things, physiologic data regarding activity occurring in the corresponding local chamber. For example, the atrial leadless device may perform sensing, including for heart sounds S1, S2, S3, or S4, and pacing operations in the right atrium, while the ventricular leadless device may perform sensing, including heart sound sensing, and pacing operations in the right ventricle.

Alternatively, leadless devices can be located in the RV or left ventricle (LV) to obtain physiologic data regarding atrial activity, including heart sounds S1, S2, S3, or S4. In addition, optionally, the leadless device could be located in the RV or LV to obtain physiologic data regarding activity in one of the LV or RV in order to determine and set a VV delay.

Alternatively, the leadless devices may be located in other chamber combinations of the heart, as well as outside of the heart. Optionally, the leadless devices may be located in a blood pool without directly engaging local tissue. Optionally, the leadless devices may be implemented solely to perform monitoring operations, without delivery of therapy. As another example, one or more leadless devices may represent a subcutaneous implantable device located in a subcutaneous pocket and configured to perform monitoring and/or deliver therapy. Optionally, the leadless devices include electrodes that are located directly on the housing of the device, without a lead extending from the device housing. Alternatively, the leadless device may be implemented with leads, where the conducted communication occurs between one or more electrodes on the lead and/or on the housing.

Embodiments herein may collect the heart sound CA signals from one or more leadless devices, analyze the heart sound CA signals and cluster the heart sound CA signals as described herein.

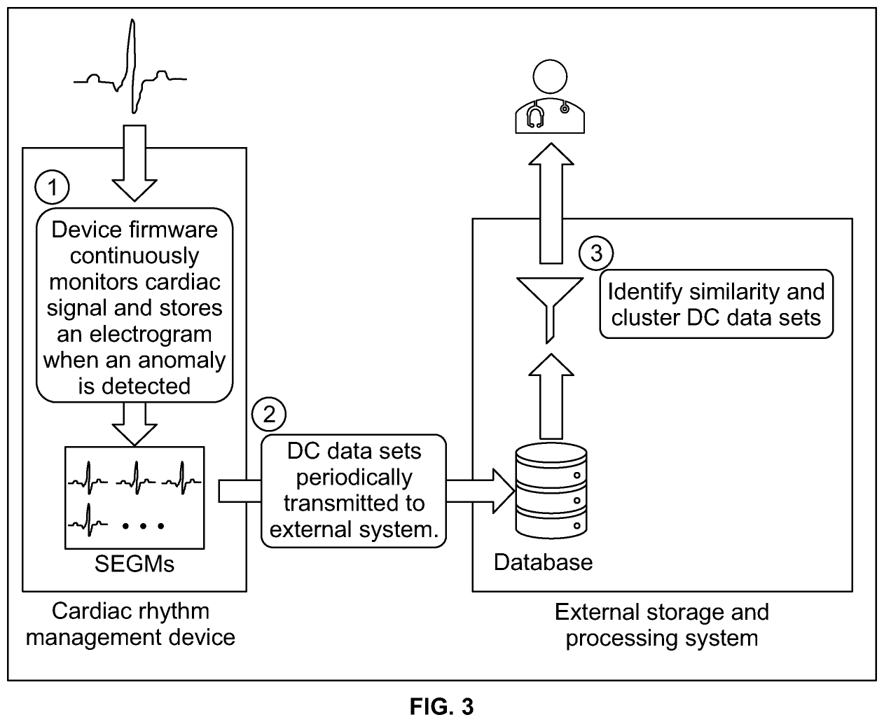

FIG. 3 shows a high-level overview of a system formed in accordance with embodiments herein. At block 1, CA signals are analyzed by one or more arrhythmia detection algorithms in the IMD. When an arrhythmia is identified, one or more DCA data sets are recorded in connection with the arrhythmia episode, including device documented markers designating characteristics of interest within the CA segment and/or identifying the nature of the arrhythmia. By way of example, one DCA data set may be recorded in connection with a single arrhythmia episode, where a single CA segment may correspond to an initial portion of the arrhythmia episode (e.g., the first 30 seconds or one minute). Additionally or alternatively, the single CA segment may correspond to another portion of the arrhythmia episode, such as the end portion of the arrhythmia episode or a segment of the arrhythmia episode exhibiting a particular characteristic of interest. The patient may experience numerous arrhythmia episodes over a day, week, month or otherwise. The IMD continuously monitors the patient's heart and records one or more DCA data sets in connection with each separate arrhythmia episode, thereby forming a collection of DCA data sets associated with a corresponding collection of arrhythmia episodes over time.

Additionally or alternatively, the IMD may also identify normal sinus rhythms and record DCNS data sets in connection there with, such as to be utilized as reference or baseline information for other analysis. Accordingly, FIG. 3 indicates, at block 2, that DC data sets are periodically transmitted to encompass both the option of transmitting DCA data sets in connection with arrhythmias and transmitting DCNS data sets in connection with normal sinus rhythms.

At various points in time, the IMD establishes a communications session with an external device, during which the opportunity arises to upload the recorded DCA data sets from the IMD to the external device, for subsequent transmission to a remote server, clinician workstation or other computing device. At block 2, the collection of DCA data sets are wirelessly transmitted from the IMD to a local external device and/or a remote server.

At block 3, the external device and/or remote server utilize one or more processes, as described herein, to compare and cluster the DC data sets, and to identify one or a subset of the DC data sets to be presented to a clinician as representative of the DCA data sets in a corresponding cluster. For example, the one or more processors may compare a current DC data set to one or more prior DC data sets in a first cluster. When the current DC data set exhibits a sufficient level of similarity to one or more of the prior DC data sets and the first cluster, the current DC data set is added to the first cluster. When an insufficient level of similarity occurs, the comparison is repeated in connection with one or more prior DC data sets in connection with a second cluster, and again with a third cluster, etc., until the current DC data set has been compared to one or more prior DC data sets in connection with each previously established cluster. In the event the current DC data set does not exhibit a sufficient level of similarity to any prior DC data set, a new cluster is created the current DC data set is assigned to the new cluster. The comparing and clustering operation group SEGMs, that are within a given similarity threshold, in a common cluster.

At block 4, the CA segment and/or information related to the CA segment for one or more DCA data sets are presented to a clinician. In accordance with embodiments herein, a representative DCA data set from each cluster is presented. For example, only the first SEGM in each cluster may be shown to the clinician. As another example, the representative DCA data set to be presented may be chosen in other manners. Rather than displaying the first CA segment as representative of a cluster, other selection criteria may be utilized. For example, the representative DCA data set may be chosen based on the longest episode duration, the most recent episode, the DCA data set that exhibits the most similarity to other DCA data sets in the cluster or the like. Additionally or alternatively, the comparing, clustering and presenting may be performed in an iterative manner and a clinician may be afforded an opportunity to change/tuned one or more thresholds that are utilized during the comparing and clustering based on the clinicians needs and various factors.

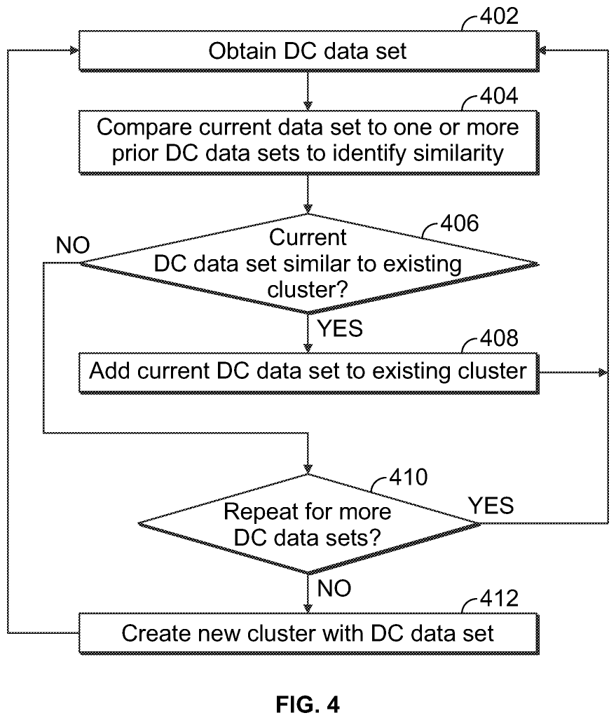

FIG. 4 illustrates a process for clustering similar DC data sets (DCA data sets and/or DCNS data sets) in accordance with embodiments herein. The operations of FIG. 4 may be implemented, in whole or in part by one or more processors of an IMD, local external device, remote server, and/or a combination thereof.

At 402, one or more processors of the system obtain a collection of DC data sets. Each of the DC data sets includes a CA segment of CA signals (e.g., EGM signals) for one or more beats. When a DC data set corresponds to a DCA data set, the individual DCA data set includes a CA segment of CA signals from a corresponding individual arrhythmia episode, and a collection of DCA data sets will correspond to a similar collection of arrhythmia episodes. For example, if a patient experiences 10 different AF episodes in one day, with each AF episode lasting 10-45 minutes, the IMD may store ten 30-60 second strips of ECG signals, with each 30-60 second strip associated with a different one of the AF episodes. Optionally, more than one 30-60 second strip of EGM signals may be recorded in connection with a single AF episode.

The CA signals are sensed along a sensing vector between a combination of electrodes. The combination of electrodes may transvenous or subcutaneous, such as when collected by a leadless IMD, transvenous IMD, subcutaneous IMD, implantable cardiac monitor and the like. that are not located transvenous. The subcutaneous electrodes may be provided on or coupled to an IMD. For example, the subcutaneous electrodes may be provided on the housing of an ICM and/or provided on a non-transvenous lead coupled to a subcutaneous IMD. Each of the DC data sets further includes one or more device documented (DD) markers, generated by the IMD, characterizing the CA signals within the corresponding DC data set.

Optionally, the DC data set may include one or more DCNS data sets for one or more reference cardiac beats known to include a corresponding a normal sinus rhythm. For example, a 30 second EGM strip may be utilized as one reference DCNS data set where the 30 second EGM strip is known to include a normal sinus rhythm. Multiple separate 30 second EGM strips may be collected at different points in time for one patient, for a patient population, recorded by a variety of device types, device placements, device orientations and the like, where the reference DCNS data sets that are known to correspond to corresponding normal rhythms.

Collection and analysis of CA segments by the IMD are initiated automatically when the IMD detects an arrhythmia episode of interest. Additionally, the IMD may collect and analyze CA signals in response to a user or clinician instruction. For example, a user or clinician may utilize a smart phone, programmer or other portable device to establish a communications session with the IMD and instruct the IMD to begin to collect and analyze cardiac signals, such as when the patient is experiencing discomfort, feeling faint, a rapid heart rate, during a clinic visit, etc.

At 404, the one or more processors compare the CA segment from a current DC data set to the CA segments from one or more prior DC data sets to identify a level of similarity there between. The one or more processors may implement the comparison in various manners. For example, the level of similarity between a pair of DC data sets may be quantified using signal processing techniques in the frequency domain. For example, a Welsh power spectral density (PSD) estimate may be calculated for the CA segment of two DC data sets. The PSD estimate calculates multiple power spectral estimates using a sliding window and then averaging the results. Optionally, a Hamming window may be applied before each transform to reduce side lobes. The PSD estimate outputs a vector of numbers representative of the power spectrum in the frequency domain for the corresponding CA segment within a corresponding DC data set. An example implementation for identifying the level of similarity is described hereafter in connection with calculating PSD.

For example, for a given two SEGM, a Welch power spectral density estimate is computed for each SEGM. Briefly, the Welch estimate is a signal processing technique that calculates multiple power spectral estimates using a sliding window and then averaging the results. A Hamming window is applied before each transform to reduce side lobe effect. Equation 1 below illustrates the overall method.

$${\hat{S}}_{x}^{W} ( {ω}_{k} ) {=}^{Δ} {\sum }_{m = 0}^{K - 1} {P}_{{x}_{m} , M} ( {ω}_{k} ) . \qquad Equation 1$$

In equation 1, K represents the number of sliding windows, and P_{xm,M}(ωₖ) represents the periodogram of each window.

The output of the Welch estimate is a vector of numbers which represents the power spectra in the frequency domain. The vector for each SEGM can be stored so that it doesn't have to be recomputed when the SEGMs are used in another comparison. Similarity between two SEGMs is then measured using the following formula:

$$Difference ( {X}_{1} , {X}_{2} ) {=}^{def} \frac{SSE}{{SSE}_{m ax}} = \frac{\sum {( {X}_{1} - {X}_{2} )}^{2}}{\sum {( max ( {X}_{1} , {X}_{2} ) )}^{2}} \qquad Equation 2$$

In equation 2, the elements X₁ and X₂ are the vectors produced by the Welch estimate, SSE is the summed squared error between the estimates, SSEₘₐₓ is the maximum possible SSE for the estimate pair, Difference ranges from 0→1. When a new SEGM is transmitted, it is compared against clusters of prior SEGMs having the same trigger. If the new SEGM X_{new} is “similar” to all SEGMs in a cluster, Clusterₙ, i.e.:

Difference(X_{new},Xₚᵣₑᵥᵢₒᵤₛ)<=α,∀Xₚᵣₑᵥᵢₒᵤₛ∈Clusterₙ

The element a represents the chosen similarity threshold. The new SEGM is added to the cluster. Otherwise a new cluster is created which contains only the new SEGM. By way of example, the earliest SEGM in each cluster is shown to the clinician. Subsequent SEGMs are filtered and/or hidden from presentation to a clinician. Optionally, the hidden SEGMs may be presented to the clinician upon request.

Additionally or alternatively, other types of similarity calculations may be performed. For example, the current and prior CA segments may be compared utilizing cross-correlation. Additionally or alternatively, the similarity calculation may be based on transitions between successive RR intervals. For example, the variation pattern in time domain between the RR intervals within the current CA segment may be compared to the variation pattern between the RR interval's within the prior CA segments of the present cluster. When a difference between the current and prior variation with a similarity threshold, the level of similarity maybe identified to be sufficient to warrant assigning the current CA segment to the present cluster. Alternatively, when a difference between the current and prior variations exceeds a threshold, the process may declare the current CA segment to be dissimilar from the prior CA segments and not justify assignment to the present cluster.

Additionally or alternatively, the one or more processors may utilize a synthetic waveform or other waveform proxy when calculating the comparison. For example, the synthetic waveform may represent a filtered version of the original CA segments, such as to remove noise, baseline drift, and other signal components not of the interest. As another example, the waveform associated with each CA segment may be simplified to primary points of transition and/or intervals of interest (e.g., intervals between R-wave markers, P-wave markers, T-wave markers, or combinations thereof).

At 406, the one or more processors determine whether the current CA segment from the corresponding current DC data set is similar to a prior CA segment from a prior DC data set within an existing cluster. For example, the one or more processors may determine a difference between the PSD for the current CA segment and a PSD for a prior CA segment. When the difference exceeds a threshold, the current and prior CA segments are deemed to be different. Alternatively, when the difference falls below the threshold, the current and prior CA segments are deemed to have a level of similarity sufficient to warrant assigning the current CA segment to the same cluster as the prior CA segment. Accordingly, when the current and prior CA segments are determined to have a sufficient level of similarity, flow moves to 408.

At 408, the one or more processors assign the current CA segment to the same cluster associated with the prior CA segment.

Alternatively, if the current CA segment does not exhibit a sufficient level of similarity to the prior CA segment presently being compared, flow moves to 410. At 410, the one or more processors determine whether additional DC data sets have yet to be analyzed and if so flow returns to 402. The determination at 410 may be a determination as to whether a present cluster has additional prior CA segments. Once all of the prior CA segments in the present cluster have been compared, the process moves to the next cluster and begin stepping through the prior CA segments in the next cluster. The operations at 402-410 are iteratively repeated until either a sufficient level of similarity is identified or all of the prior CA segments are analyzed and no sufficient level of similarity is identified.

At 410, when the one or more processors determine that the current CA segment is not similar to any prior CA segment, flow moves to 412. At 412, the one or processors create a new cluster and assigns the current CA segment to the new cluster.

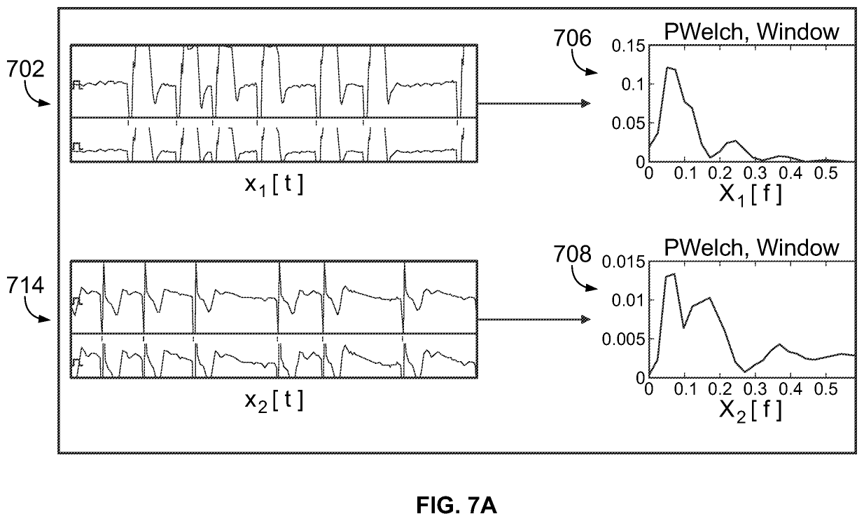

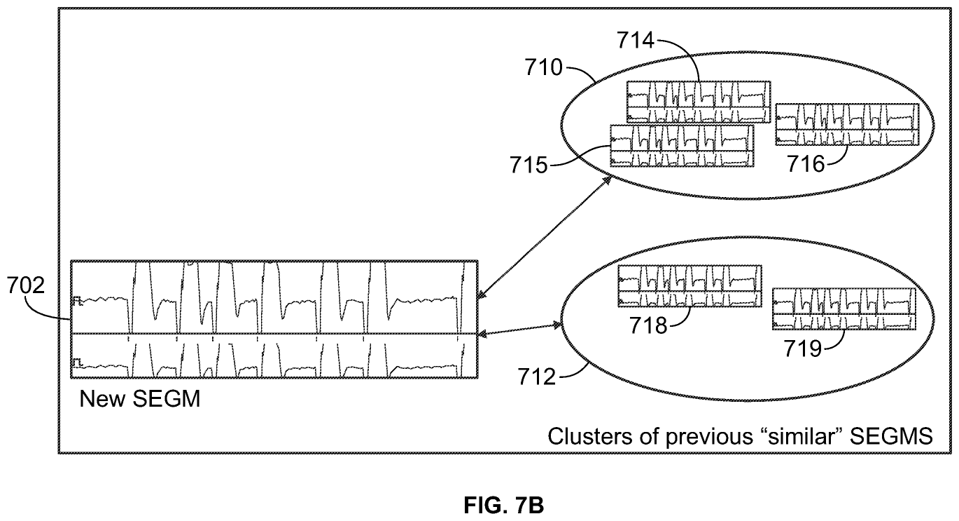

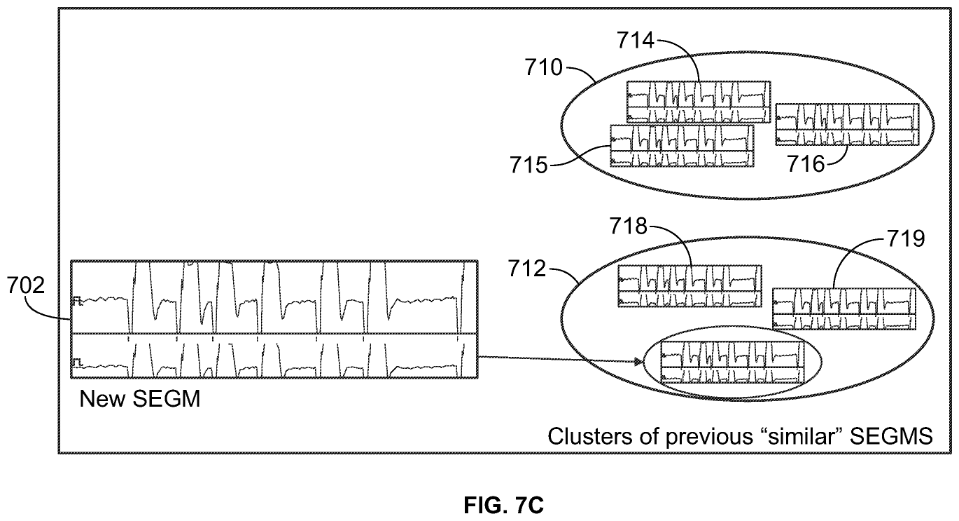

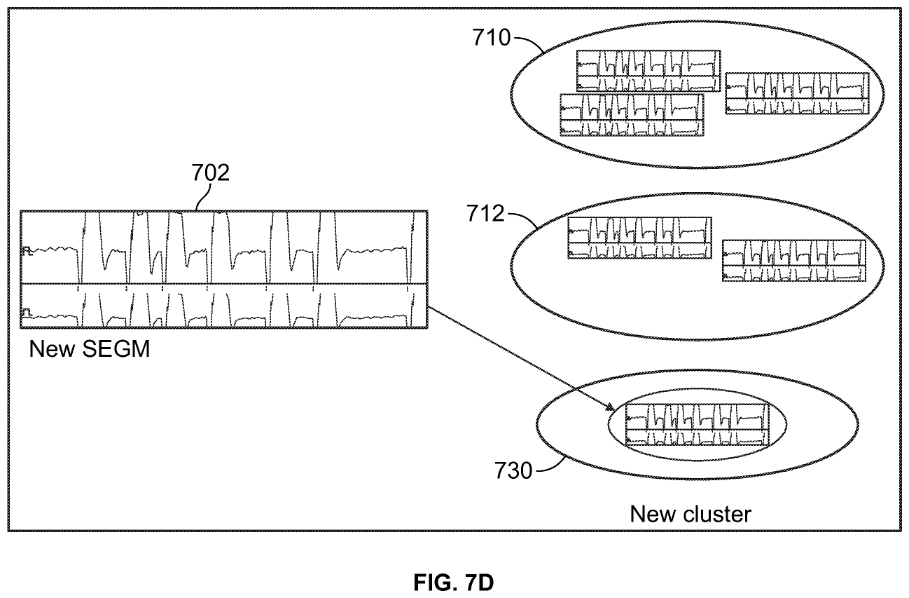

To further illustrate the similarity determination, reference is made to FIGS. 7A-7D. FIG. 7A illustrates first and second CA segments 702, 714 representing CA signals in the time domain. The CA segment 714 may represent a prior CA segment that has already been analyzed and assigned to a cluster, such as a first cluster, while the CA segment 702 may represent a new or current CA segment to be grouped in a cluster. As explained in connection with FIG. 4, the CA segments 702 and 714 are converted to frequency domain CA segments 706 and 708, respectively. The frequency domain CA segments 706 and 708 are compared for a level of similarity.

FIG. 7B illustrates first and second clusters 710, 712, into which prior CA segments have been assigned. The first cluster 710 includes prior CA segments 714-716, while the second clusters 712 includes prior CA segments 718, 719. The current CA segment 702 is compared to the prior CA segment 714. When a sufficient level of similarity is identified, the new CA segment 702 is assigned to the first cluster 710 and the process ends. Alternatively, when a sufficient level of similarity is not identified, the current CA segment 702 is compared to the next prior CA segment 715, and thereafter (if necessary) to the next prior CA segment 716. If the level of similarity does not satisfy the similarity threshold, the process determines that the CA segment 702 should not be assigned to the first cluster 710. Thereafter, the process is repeated to compare the current CA segment 702 to one or more of the prior CA segment 718, 719 and the second cluster 712.

As shown at FIG. 7C, when the current CA segment 702 is identified to be similar to at least one of the prior CA segments 718, 719, the current CA segment 702 is assigned to the second cluster 712. Alternatively, as shown in connection with FIG. 7D, when the current CA segment 702 does not exhibit sufficient similarity to the CA segments 718, 719, given that no other clusters exist, a new cluster is created at cluster 730 and the current CA segment 702 is assigned to the new cluster 730.

Additionally or alternatively, the determination of which cluster to assign the current CA segment may be determined based solely utilizing machine learning algorithms, or in combination with machine learning algorithms. For example, a machine learning algorithm may be applied to compare a current CA segment with prior CA segments to assign the clustering. Additionally or alternatively, the cluster assignment may be based in part on “key episodes”. For example, certain characteristics of interest may be defined such that, all CA segments having the characteristics of interest are assigned to a common cluster.

In accordance with the operations of FIGS. 4 and 7, one or more processors are configured to execute the specific executable instructions to: obtain DC data sets generated by an implantable medical device, the DC data sets including a corresponding cardiac activity (CA) segment from an episode identified by the IMD; compare the CA segments, associated with different episodes, to one another to identify a level of similarity therebetween; separate the CA segments into at least first and second clusters based on the level of similarity; designate a first representative CA segment from the first cluster to be representative of the CA segments in the first cluster; and designate a second representative CA segment from the second cluster to be representative of the CA segments in the second cluster; and a display to present the first and second representative CA segments as representative of the first and second clusters. For example, the first representative CA segment is associated with a first episode. The first cluster includes additional CA segments, associated with additional episodes. The additional CA segments fall within the level of similarity to the first representative CA segment.

The clusters 710, 712 and 730 includes additional CA segments that are redundant as to shape, morphology and/or other characteristic of interest of the first representative CA segment from each of the clusters 710, 712 and 730. In accordance with embodiments herein, the one or more processors are configured to not display the additional CA segments.

Additionally or alternatively, when determining which representative CA segment to display, the one or more processors may be further configured to select, as the representative CA segment for a given cluster, a one of the CA segments in the given cluster that at least one of: i) was first assigned to the first cluster, ii) the associated episode exhibits a longest duration, iii) was the most recently assigned to the cluster, or iv) exhibits a select level of similarity to a remainder of the CA segments in the first cluster.

Next, the discussion turns to an example implementation for identifying a level of similarity between DC data sets.

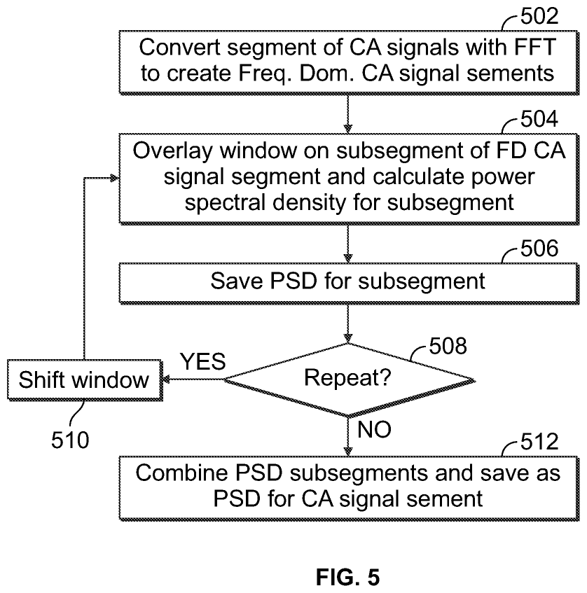

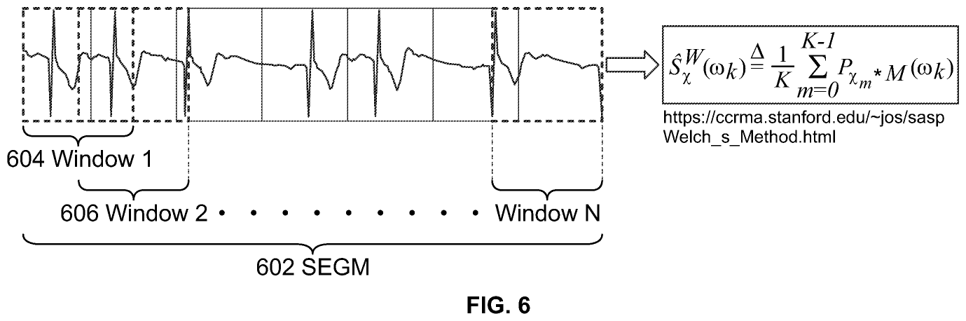

FIG. 5 illustrates a process for calculating PSD estimates for a CA segment from a single DC data set in accordance with embodiments herein. At 502, the one or more processors apply a discrete Fourier transform to the CA signals to create a frequency domain (FD) CA segment. FIG. 6 illustrates a graphical example of a manner in which CA signals may be converted to the frequency domain. FIG. 6 illustrates a CA segment 602 (e.g., a 30-60 second strip of stored EGM (SEGM) signals) in the time domain plotting voltage along a vertical axis and time along the horizontal axis.

Returning to FIG. 5, at 504, the one or more processors overlay a window onto a subsegment of the FD CA segment. In FIG. 6, the window may correspond to “window 1” at 604. At 504, the one or more processors calculate a power spectral density within the subsegment corresponding to window 604.

At 506, the one or more processors save the PSD for the corresponding subsegment 604. At 508, the one or more processors determine whether the FD CA segment has been entirely analyzed. If not, flow moves to 510. At 510, the one or more processors shift the window to a next subsegment. With reference to FIG. 6, the window may be shifted to the position denoted by “window 2” at 606. The amount of shift may vary. For example, the window may be shifted between the window 1 at 604 and the window 2 at 606 such as by moving the window in time 25%, 50%, 75% of the full width of the window. Next, at 504, the one or more processors calculate the PSD for the next subsegment corresponding to window 606. The operations at 504-510 are repeated until the entire FD CA segment 602 has been analyzed to derive a corresponding vector of PSD values.

At 512, the PSD values for the subsegments are combined and saved as an overall PSD for the CA segment. The overall PSD may be defined as a vector with each element of the vector corresponding to one of the subsegment/windows 604, 606, etc. Additionally or alternatively, the overall PSD may represent a mathematical combination of the PSDs for the individual subsegments (e.g., an average).

Additionally or alternatively, embodiments herein may be applied to reduce the possibility of true positive CA segments being missed or otherwise filtered out of presentation to a clinician. In connection there with, new/current CA segments may be presented to a clinician, even if determined to be within the level of similarity to prior CA segments, when a select period of time (e.g., a number of days) have passed between a current CA segment and a most recent prior CA segment in the same cluster. Additionally or alternatively, new/current CA segments may be presented to a clinician, even if determined to be within the level of similarity to prior CA segments, when a cluster reaches a predetermined size. For example, when a cluster recent predetermined size, a series of representatives CA segments maybe identified from the cluster, such as the oldest, an intermediate and the newest CA segments.

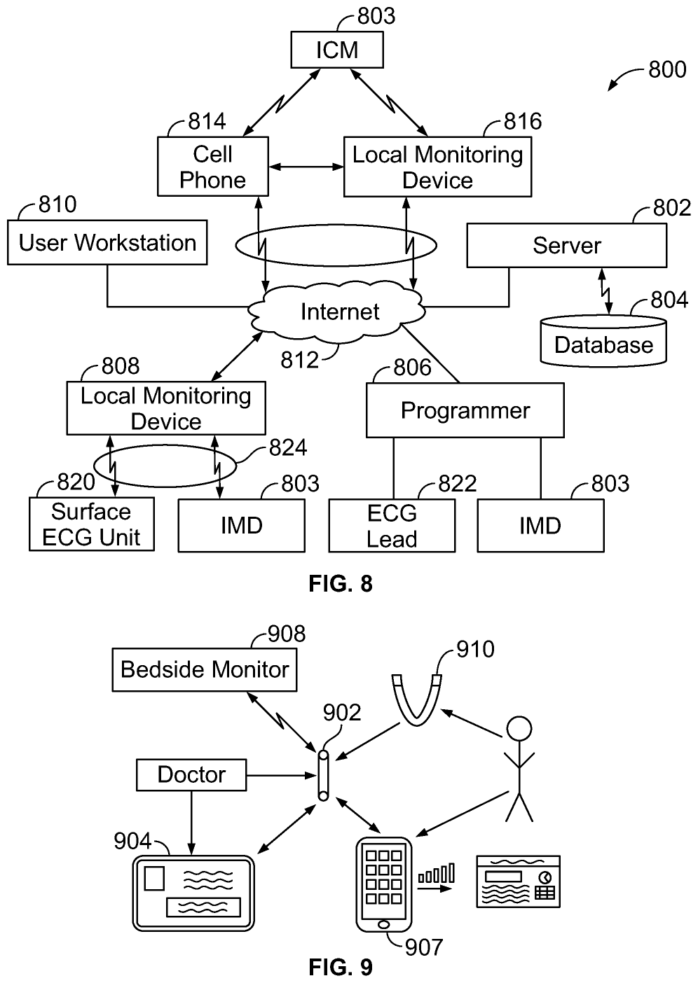

FIG. 8 illustrates a distributed processing system 800 in accordance with embodiments herein. The distributed processing system 800 includes a server 802 connected to a database 804, a programmer 806, a local monitoring device 808 (e.g., IMD 100) and a user workstation 810 electrically connected to a network 812. Any of the processor-based components in FIG. 6 (e.g., workstation 810, cell phone 814, local monitoring device 816, server 802, programmer 806) may perform the processes discussed herein.

The network 812 may provide cloud-based services over the internet, a voice over IP (VoIP) gateway, a local plain old telephone service (POTS), a public switched telephone network (PSTN), a cellular phone-based network, and the like. Alternatively, the communication system may be a local area network (LAN), a medical campus area network (CAN), a metropolitan area network (MAN), or a wide area network (WAM). The communication system serves to provide a network that facilitates the transfer/receipt of data and other information between local and remote devices (relative to a patient). The server 802 is a computer system that provides services to the other computing devices on the network 812. The server 802 controls the communication of information such as DCA data sets, CA signals, motion data, bradycardia episode information, asystole episode information, arrythmia episode information, markers, heart rates, and device settings. The server 802 interfaces with the network 812 to transfer information between the programmer 806, local monitoring devices 808, 816, user workstation 810, cell phone 814 and database 804. For example, the server 802 may receive DCA data sets from various clinics, medical networks, individual patient's and the like. The server 802 may further push new comparison and clustering models and/or updated versions of models to various other devices, such as the programmers, local monitoring devices, cell phones, workstations and the like illustrated in FIG. 8. The database 804 stores information such as DC data sets, arrythmia episode information, arrythmia statistics, diagnostics, DD markers, CA signal, heart rates, device settings, and the like, for a patient population, as well as separated for individual patients, individual physicians, individual clinics, individual medical networks and the like. The server 802 may implement the operations described in connection with FIGS. 3-7.

The programmer 806 may reside in a patient's home, a hospital, or a physician's office. The programmer 806 may wirelessly communicate with the IMD 803 and utilize protocols, such as Bluetooth, GSM, infrared wireless LANs, HIPERLAN, 3G, satellite, as well as circuit and packet data protocols, and the like. Alternatively, a telemetry “wand” connection may be used to connect the programmer 806 to the IMD 803. The programmer 806 is able to acquire ECG 822 from surface electrodes on a person (e.g., ECGs), electrograms (e.g., EGM) signals from the IMD 803, and/or CA data, arrythmia episode information, arrythmia statistics, diagnostics, markers, CA signal waveforms, atrial heart rates, device settings from the IMD 803. The programmer 806 interfaces with the network 812, either via the internet, to upload the information acquired from the surface ECG unit 820, or the IMD 803 to the server 802.

The local monitoring device 808 interfaces with the communication system to upload to the server 802 one or more of the DC data sets, CA segments, motion data, arrythmia episode information, arrythmia statistics, diagnostics, markers, heart rates, sensitivity profile parameter settings and detection thresholds. In one embodiment, the surface ECG unit 820 and the IMD 803 have a bi-directional connection 824 with the local RF monitoring device 808 via a wireless connection. The local monitoring device 808 is able to acquire surface ECG signals from an ECG lead 822, as well as DCA CA data sets and other information from the IMD 803. On the other hand, the local monitoring device 808 may download the data and information discussed herein from the database 804 to the IMD 803.

The user workstation 810, cell phone 814 and/or programmer 806 may be utilized by a physician or medical personnel to interface with the network 812 to download DCA data sets, CA signals, motion data, and other information discussed herein from the database 804, from the local monitoring devices 808, 816, from the IMD 803 or otherwise. Once downloaded, the user workstation 810 may process the DC data sets, CA signals and motion data in accordance with one or more of the operations described above.

For example, one or more processors of the various computing devices in FIG. 8 may be configured to: obtain device classified (DC) data sets generated by an implantable medical device (IMD), each of the DC data sets including a cardiac activity (CA) segment from an episode identified by the IMD; compare the CA segments, associated with different episodes, to one another to identify a level of similarity therebetween; separate the CA segments into at least first and second clusters based on the level of similarity; designate a first representative CA segment from the first cluster to be representative of the CA segments in the first cluster; and designate a second representative CA segment from the second cluster to be representative of the CA segments in the second cluster. One or more displays from the various computing devices in FIG. 8 may be configured to present the first and second representative CA segments as representative of the first and second clusters.

Additionally or alternatively, the first representative CA segment is associated with a first episode, the first cluster includes additional CA segments, associated with additional episodes, the additional CA segments falling within the level of similarity to the first representative CA segment. Additionally or alternatively, the first cluster includes additional CA segments are redundant as to shape, morphology and/or other characteristic of interest of the first representative CA segment, the one or more processors is further configured to not display the additional CA signals. Additionally or alternatively, the one or more processors are configured to calculate a power spectral density (PSD) for each of the CA segments and to compare the PSDs for respective ones of the CA segments to identify the level of similarity. Additionally or alternatively, the one or more processors are configured to compare the CA signals by utilizing at least one of a cross correlation technique or a power spectral estimate. Additionally or alternatively, the first and second clusters include prior first and second sets of CA segments, the one or more processors further configured to compare a current CA segment to the prior first set of CA segments, and if the level of similarity does not satisfy the threshold, to then compare the current CA segments to the prior second set of CA segments. Additionally or alternatively, the one or more processors are further configured to select, as the first representative CA segment, a one of the CA segments in the first cluster that at least one of: i) was first assigned to the first cluster, ii) exhibits a longest duration, iii) was the most recently assigned to the cluster, or iv) exhibits a select level of similarity to a remainder of the CA segments in the first cluster.

The user workstation 810, cell phone 814 and/or programmer 806, may be used to present information, such as the representative CA segments associated with each cluster. The devices may further be configured to present more than one representative CA segment in connection with each cluster, although not all CA segments associated with any cluster need be displayed. The user workstation 810, cell phone 814 and/or programmer 806 may upload/push settings (e.g., sensitivity profile parameter settings), IMD instructions, other information and notifications to the cell phone 814, local monitoring devices 808, 816, programmer 806, server 802 and/or IMD 803.

The processes described herein in connection with reducing false positive arrhythmias may be performed by one or more of the devices illustrated in FIG. 8, including but not limited to the IMD 803, programmer 806, local monitoring devices 808, 816, user workstation 810, cell phone 814, and server 802. The process described herein may be distributed between the devices of FIG. 8. For example, one or more of the devices illustrated in FIG. 8 may include memory to store specific executable instructions and a machine learning (ML) model; and one or more processors configured to execute the specific executable instructions to perform the operations described herein.

FIG. 8 comprise a combination of subcutaneous electrodes configured to collect the CA signals; IMD memory configured to store program instructions; and one or more IMD processors configured to execute the program instructions to: analyze the CA signals and based on the analysis declare candidate arrhythmias episodes; generate the DCA data sets including the corresponding CA segment and the corresponding DD markers; and a transceiver configured to wirelessly transmit the DCA data sets to an external device. One or more of the external devices in FIG. 8 include the memory and the one or more processors and a transceiver, the transceiver configured to wirelessly receive the DCA data sets from the IMD. The server 802 may include memory and the one or more processors, the memory configured to store the collection of the DCA data sets, the one or more processors configured to perform the comparing, separating, designating and presenting operations described herein.

FIG. 9 illustrates a system level diagram indicating potential devices and networks that utilize the methods and systems herein. For example, an IMD 902 may be utilized to collect a DCA data set. The IMD 902 may supply the DCA data set (CA segments, DD markers) as well as sensitivity levels and motion data, to various local external devices, such as a tablet device 904, a smart phone 906, a bedside monitoring device 908, a smart watch and the like. The devices 904-908 include a display to present the various types of the CA segments, DD markers, statistics (e.g., % valid, % invalid), diagnostics, recommendations for adjustments in IMD sensing/therapy parameters and other information described herein. The IMD 902 may convey the DCA data set over various types of wireless communications links to the devices 904, 906 and 908. The IMD 902 may utilize various communications protocols and be activated in various manners, such as through a Bluetooth, Bluetooth low energy, Wi-Fi or other wireless protocol. Additionally or alternatively, when a magnetic device 910 is held next to the patient, the magnetic field from the device 910 may activate the IMD 902 to transmit the DCA data set and arrythmia data to one or more of the devices 904-908.

The foregoing embodiments are described primarily in connection with electrical CA signals, it is recognized that the CA signals may also be from other sources such as impedance measurements, heart sound measurements and the like.

Embodiments may be implemented in connection with one or more implantable medical devices (IMDs). Non-limiting examples of IMDs include one or more of implantable leadless monitoring and/or therapy devices, and/or alternative implantable medical devices. For example, the IMD may represent a cardiac monitoring device, pacemaker, cardioverter, cardiac rhythm management device, defibrillator, leadless monitoring device, leadless pacemaker and the like. Additionally or alternatively, the IMD may be a leadless implantable medical device (LIMD) that include one or more structural and/or functional aspects of the device(s) described in U.S. Pat. No. 9,216,285 “LEADLESS IMPLANTABLE MEDICAL DEVICE HAVING REMOVABLE AND FIXED COMPONENTS” and U.S. Pat. No. 8,831,747 “LEADLESS NEUROSTIMULATION DEVICE AND METHOD INCLUDING THE SAME”, which are hereby incorporated by reference. Additionally or alternatively, the IMD may include one or more structural and/or functional aspects of the device(s) described in U.S. Pat. No. 8,391,980 “METHOD AND SYSTEM FOR IDENTIFYING A POTENTIAL LEAD FAILURE IN AN IMPLANTABLE MEDICAL DEVICE” and U.S. Pat. No. 9,232,485 “SYSTEM AND METHOD FOR SELECTIVELY COMMUNICATING WITH AN IMPLANTABLE MEDICAL DEVICE”, which are hereby incorporated by reference. Additionally or alternatively, the IMD may be a subcutaneous IMD that includes one or more structural and/or functional aspects of the device(s) described in U.S. application Ser. No. 15/973,195, titled “SUBCUTANEOUS IMPLANTATION MEDICAL DEVICE WITH MULTIPLE PARASTERNAL-ANTERIOR ELECTRODES” and filed May 7, 2018; U.S. application Ser. No. 15/973,219, titled “IMPLANTABLE MEDICAL SYSTEMS AND METHODS INCLUDING PULSE GENERATORS AND LEADS” filed May 7, 2018; U.S. application Ser. No. 15/973,249, titled “SINGLE SITE IMPLANTATION METHODS FOR MEDICAL DEVICES HAVING MULTIPLE LEADS”, filed May 7, 2018, which are hereby incorporated by reference in their entireties. Additionally or alternatively, the IMD may be a leadless cardiac monitor (ICM) that includes one or more structural and/or functional aspects of the device(s) described in U.S. Patent Application having Docket No. A15E1059, U.S. patent application Ser. No. 15/084,373, filed Mar. 29, 2016, entitled, “METHOD AND SYSTEM TO DISCRIMINATE RHYTHM PATTERNS IN CARDIAC ACTIVITY,”; U.S. patent application Ser. No. 15/973,126, titled “METHOD AND SYSTEM FOR SECOND PASS CONFIRMATION OF DETECTED CARDIAC ARRHYTHMIC PATTERNS”; U.S. patent application Ser. No. 15/973,351, titled “METHOD AND SYSTEM TO DETECT R-WAVES IN CARDIAC ARRHYTHMIC PATTERNS”; U.S. patent application Ser. No. 15/973,307, titled “METHOD AND SYSTEM TO DETECT POST VENTRICULAR CONTRACTIONS IN CARDIAC ARRHYTHMIC PATTERNS”; U.S. patent application Ser. No. 16/399,813, titled “METHOD AND SYSTEM TO DETECT NOISE IN CARDIAC ARRHYTHMIC PATTERNS”; and U.S. patent application Ser. No. 16/930,791, filed Jul. 16, 2020, titled “METHODS, DEVICES AND SYSTEMS FOR HOLISTIC INTEGRATED HEALTHCARE PATIENT MANAGEMENT”, which are hereby incorporated by reference. Further, one or more combinations of IMDs may be utilized from the above incorporated patents and applications in accordance with embodiments herein.

### CLOSING

The various methods as illustrated in the Figures and described herein represent exemplary embodiments of methods. The methods may be implemented in software, hardware, or a combination thereof. In various of the methods, the order of the steps may be changed, and various elements may be added, reordered, combined, omitted, modified, etc. Various of the steps may be performed automatically (e.g., without being directly prompted by user input) and/or programmatically (e.g., according to program instructions).

Various modifications and changes may be made as would be obvious to a person skilled in the art having the benefit of this disclosure. It is intended to embrace all such modifications and changes and, accordingly, the above description is to be regarded in an illustrative rather than a restrictive sense.

Various embodiments of the present disclosure utilize at least one network that would be familiar to those skilled in the art for supporting communications using any of a variety of commercially-available protocols, such as Transmission Control Protocol/Internet Protocol (“TCP/IP”), User Datagram Protocol (“UDP”), protocols operating in various layers of the Open System Interconnection (“OSI”) model, File Transfer Protocol (“FTP”), Universal Plug and Play (“UpnP”), Network File System (“NFS”), Common Internet File System (“CIFS”) and AppleTalk. The network can be, for example, a local area network, a wide-area network, a virtual private network, the Internet, an intranet, an extranet, a public switched telephone network, an infrared network, a wireless network, a satellite network and any combination thereof.

In embodiments utilizing a web server, the web server can run any of a variety of server or mid-tier applications, including Hypertext Transfer Protocol (“HTTP”) servers, FTP servers, Common Gateway Interface (“CGI”) servers, data servers, Java servers, Apache servers and business application servers. The server(s) also may be capable of executing programs or scripts in response to requests from user devices, such as by executing one or more web applications that may be implemented as one or more scripts or programs written in any programming language, such as Java®, C, C# or C++, or any scripting language, such as Ruby, PHP, Perl, Python or TCL, as well as combinations thereof. The server(s) may also include database servers, including without limitation those commercially available from Oracle®, Microsoft®, Sybase®, SAS® and IBM® as well as open-source servers such as MySQL, Postgres, SQLite, MongoDB, and any other server capable of storing, retrieving and accessing structured or unstructured data. Database servers may include table-based servers, document-based servers, unstructured servers, relational servers, non-relational servers or combinations of these and/or other database servers.

The environment can include a variety of data stores and other memory and storage media as discussed above. These can reside in a variety of locations, such as on a storage medium local to (and/or resident in) one or more of the computers or remote from any or all of the computers across the network. In a particular set of embodiments, the information may reside in a storage-area network (“SAN”) familiar to those skilled in the art. Similarly, any necessary files for performing the functions attributed to the computers, servers or other network devices may be stored locally and/or remotely, as appropriate. Where a system includes computerized devices, each such device can include hardware elements that may be electrically coupled via a bus, the elements including, for example, at least one central processing unit (“CPU” or “processor”), at least one input device (e.g., a mouse, keyboard, controller, touch screen or keypad) and at least one output device (e.g., a display device, printer or speaker). Such a system may also include one or more storage devices, such as disk drives, optical storage devices and solid-state storage devices such as random access memory (“RAM”) or read-only memory (“ROM”), as well as removable media devices, memory cards, flash cards, etc.

Such devices also can include a computer-readable storage media reader, a communications device (e.g., a modem, a network card (wireless or wired), an infrared communication device, etc.) and working memory as described above. The computer-readable storage media reader can be connected with, or configured to receive, a computer-readable storage medium, representing remote, local, fixed and/or removable storage devices as well as storage media for temporarily and/or more permanently containing, storing, transmitting and retrieving computer-readable information. The system and various devices also typically will include a number of software applications, modules, services or other elements located within at least one working memory device, including an operating system and application programs, such as a client application or web browser. It should be appreciated that alternate embodiments may have numerous variations from that described above. For example, customized hardware might also be used and/or particular elements might be implemented in hardware, software (including portable software, such as applets) or both. Further, connection to other computing devices such as network input/output devices may be employed.

Various embodiments may further include receiving, sending, or storing instructions and/or data implemented in accordance with the foregoing description upon a computer-readable medium. Storage media and computer readable media for containing code, or portions of code, can include any appropriate media known or used in the art, including storage media and communication media, such as, but not limited to, volatile and non-volatile, removable and non-removable media implemented in any method or technology for storage and/or transmission of information such as computer readable instructions, data structures, program modules or other data, including RAM, ROM, Electrically Erasable Programmable Read-Only Memory (“EEPROM”), flash memory or other memory technology, Compact Disc Read-Only Memory (“CD-ROM”), digital versatile disk (DVD) or other optical storage, magnetic cassettes, magnetic tape, magnetic disk storage or other magnetic storage devices or any other medium which can be used to store the desired information and which can be accessed by the system device. Based on the disclosure and teachings provided herein, a person of ordinary skill in the art will appreciate other ways and/or methods to implement the various embodiments.

The specification and drawings are, accordingly, to be regarded in an illustrative rather than a restrictive sense. It will, however, be evident that various modifications and changes may be made thereunto without departing from the broader spirit and scope of the invention as set forth in the claims.

Other variations are within the spirit of the present disclosure. Thus, while the disclosed techniques are susceptible to various modifications and alternative constructions, certain illustrated embodiments thereof are shown in the drawings and have been described above in detail. It should be understood, however, that there is no intention to limit the invention to the specific form or forms disclosed, but on the contrary, the intention is to cover all modifications, alternative constructions and equivalents falling within the spirit and scope of the invention, as defined in the appended claims.

The use of the terms “a” and “an” and “the” and similar referents in the context of describing the disclosed embodiments (especially in the context of the following claims) are to be construed to cover both the singular and the plural, unless otherwise indicated herein or clearly contradicted by context. The terms “comprising,” “having,” “including” and “containing” are to be construed as open-ended terms (i.e., meaning “including, but not limited to,”) unless otherwise noted. The term “connected,” when unmodified and referring to physical connections, is to be construed as partly or wholly contained within, attached to or joined together, even if there is something intervening. Recitation of ranges of values herein are merely intended to serve as a shorthand method of referring individually to each separate value falling within the range, unless otherwise indicated herein and each separate value is incorporated into the specification as if it were individually recited herein. The use of the term “set” (e.g., “a set of items”) or “subset” unless otherwise noted or contradicted by context, is to be construed as a nonempty collection comprising one or more members. Further, unless otherwise noted or contradicted by context, the term “subset” of a corresponding set does not necessarily denote a proper subset of the corresponding set, but the subset and the corresponding set may be equal.

Operations of processes described herein can be performed in any suitable order unless otherwise indicated herein or otherwise clearly contradicted by context. Processes described herein (or variations and/or combinations thereof) may be performed under the control of one or more computer systems configured with executable instructions and may be implemented as code (e.g., executable instructions, one or more computer programs or one or more applications) executing collectively on one or more processors, by hardware or combinations thereof. The code may be stored on a computer-readable storage medium, for example, in the form of a computer program comprising a plurality of instructions executable by one or more processors. The computer-readable storage medium may be non-transitory.

All references, including publications, patent applications and patents, cited herein are hereby incorporated by reference to the same extent as if each reference were individually and specifically indicated to be incorporated by reference and were set forth in its entirety herein.

It is to be understood that the subject matter described herein is not limited in its application to the details of construction and the arrangement of components set forth in the description herein or illustrated in the drawings hereof. The subject matter described herein is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions, types of materials and physical characteristics described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. § 112(f), unless and until such claim limitations expressly use the phrase “means for” followed by a statement of function void of further structure.

What is claimed is:

1. A system for managing presentation of cardiac activity signals, comprising:
memory to store specific executable instructions;
one or more processors configured to execute the specific executable instructions to:
obtain device classified (DC) data sets generated by an implantable medical device (IMD), the DC data sets including a corresponding cardiac activity (CA) segment from an episode identified by the IMD;
compare the CA segments, associated with different episodes, to one another to identify a level of similarity therebetween;
separate the CA segments into at least first and second clusters based on the level of similarity;
designate a first representative CA segment from the first cluster to be representative of the CA segments in the first cluster; and
designate a second representative CA segment from the second cluster to be representative of the CA segments in the second cluster; and
a display to present the first and second representative CA segments as representative of the first and second clusters.

2. The system of claim 1, wherein the first representative CA segment is associated with a first episode, the first cluster includes additional CA segments, associated with additional episodes, the additional CA segments falling within the level of similarity to the first representative CA segment.

3. The system of claim 1, wherein the first cluster includes additional CA segments are redundant as to shape, morphology and/or other characteristic of interest of the first representative CA segment, the one or more processors further configured to not display the additional CA segments.

4. The system of claim 1, wherein the one or more processors are configured to calculate a power spectral density (PSD) for each of the CA segments and to compare the PSDs for respective ones of the CA segments to identify the level of similarity.

5. The system of claim 1, wherein the one or more processors are configured to compare the CA signals by utilizing at least one of a cross correlation technique or a power spectral estimate.

6. The system of claim 1, wherein the first and second clusters include prior first and second sets of CA segments, the one or more processors further configured to compare a current CA segment to the prior first set of CA segments, and if the level of similarity does not satisfy the threshold, to then compare the current CA segments to the prior second set of CA segments.

7. The system of claim 1, wherein the one or more processors are further configured to select, as the first representative CA segment, a one of the CA segments in the first cluster that at least one of: i) was first assigned to the first cluster, ii) associated with the longest episode duration, iii) was the most recently assigned to the cluster, or iv) exhibits a select level of similarity to a remainder of the CA segments in the first cluster.

8. The system of claim 1, further comprising a sensor to collect CA signals, the DC data set based on the CA signals, the CA signals indicative of at least one of impedance, electrical or mechanical activity by one or more heart chambers or by a local region within the heart.

9. The system of claim 9, wherein the CA signals includes at least one of EGM signals or heart sound (HS) based CA signals, the HS based CA signals indicative of one or more of the S1, S2, S3 or S4 heart sounds.

10. A computer implemented method, comprising:
under control of one or more processors configured with specific executable instructions,
obtaining device classified (DC) data sets generated by an implantable medical device (IMD), each of the DC data sets including a cardiac activity (CA) segment from an episode identified by the IMD;
comparing the CA segments, associated with different episodes, to one another to identify a level of similarity therebetween;
separating the CA segments into at least first and second clusters based on the level of similarity;
designating a first representative CA segment from the first cluster to be representative of the CA segments in the first cluster; and
designating a second representative CA segment from the second cluster to be representative of the CA segments in the second cluster; and
presenting the first and second representative CA segments as representative of the first and second clusters.

11. The method of claim 10, wherein the first representative CA segment is associated with a first episode, the first cluster includes additional CA segments, associated with additional episodes, the additional CA segments falling within the level of similarity to the first representative CA segment.

12. The method of claim 10, wherein the first cluster includes additional CA segments are redundant as to shape, morphology and/or other characteristic of interest of the first representative CA segment, the method further comprising not displaying the additional CA signals.

13. The method of claim 10, further comprising calculating a power spectral density (PSD) for each of the CA segments and comparing the PSDs for respective ones of the CA segments to identify the level of similarity.

14. The method of claim 10, wherein the comparing the CA signals utilizes at least one of a cross correlation technique or a power spectral estimate.

15. The method of claim 10, wherein the first and second clusters include prior first and second sets of CA segments, the method further comprising comparing a current CA segment to the prior first set of CA segments, and if the level of similarity does not satisfy the threshold, then comparing the current CA segments to the prior second set of CA segments.

16. The method of claim 10, further comprising selecting, as the first representative CA segment, a one of the CA segments in the first cluster that at least one of: i) was first assigned to the first cluster, ii) associated with the longest episode duration, iii) was the most recently assigned to the cluster, or iv) exhibits a select level of similarity to a remainder of the CA segments in the first cluster.

17. The method of claim 10, further comprising utilizing a sensor to collect CA signals, the DC data set based on the CA signals, the CA signals indicative of at least one of impedance, electrical or mechanical activity by one or more heart chambers or by a local region within the heart.

18. The method of claim 17, wherein the CA signals includes at least one of EGM signals or heart sound (HS) based CA signals, the HS based CA signals indicative of one or more of the S1, S2, S3 or S4 heart sounds.
